(19) United States
(12) Patent Application Publication
(10) Pub. No.: US 2022/0257116 A1
(43) Pub. Date: Aug. 18, 2022

(54) FUNDUS IMAGE PHOTOGRAPHY APPARATUS HAVING AUTOMATIC FOCUSING FUNCTION

(71) Applicant: UMI OPTICS, Seoul (KR)

(72) Inventor: Dong-Kwan KIM, Namyangju-si (KR)

(73) Assignee: UMI OPTICS, Seoul (KR)

(21) Appl. No.: 17/631,203

(22) PCT Filed: Jul. 29, 2020
(86) PCT No.: PCT/KR2020/010007

§ 371 Date: Mar. 25, 2022

(30) Foreign Application Priority Data
Jul. 29, 2019 (KR) 10-2019-0091518

(51) Int. Cl. A61B 3/14; A61B 3/12; A61B 3/00; G03B 13/36

(52) U.S. Cl. CPC A61B 3/14; G03B 13/36; A61B 3/0008; A61B 3/12

(57) ABSTRACT

The present invention relates to a fundus imaging device having an automatic focusing function, by which even a user, who is not a skilled expert, may easily photograph a fundus image, and interpret and analyze the photographed image to predict a degree of risk, the fundus imaging device controlling a focusing unit to adjust a focus of a pupil image received from a pupil imaging unit, and controlling a fundus imaging unit to photograph a fundus image when the pupil image is in focus.

### TECHNICAL FIELD

The present invention relates to a fundus imaging device having an automatic focusing function, and more particularly, to a fundus imaging device having an automatic focusing function, by which even a user, who is not a skilled expert, may easily photograph a fundus image, and interpret and analyze the photographed image to predict a degree of risk.

### BACKGROUND ART

Examining human eyeball and fundus allows interpretation of information on a specific body part, and through this information, the health condition of the body or the presence or absence of diseases may be readily diagnosed. Recently, various diseases and genetic disorder are diagnosed by taking pictures of a fundus by using a fundus imaging device equipped with a camera and analyzing the photographed images.

Commonly used fundus imaging devices are largely installed and used in hospitals, and examples thereof may include slit lamp microscopes, fundus cameras, and meibomian gland measuring apparatuses. Such fundus imaging devices may examine anterior segment, crystalline lens, retina, inner eyelid, and the like to diagnose eye diseases such as cataract, glaucoma, diabetic retinopathy, age-related macular degeneration, and obstructive meibomian gland dysfunction. Regarding a fundus imaging method using such a fundus imaging device, for example, an examinee is positioned in front of the fundus imaging device, and after setting a distance between the imaging device and a cornea of the examinee and aligning an image formation of a pupil of the examinee, a retina is observed and a retina image is captured after adjusting a focus on the retina in a state that the distance to the cornea and the alignment with the pupil are maintained.

However, in a conventional fundus imaging device, it is necessary to provide an illumination optical system for providing illumination, an imaging optical system for transmitting light reflected from the fundus as an imaging light, a surface projection optical system for projecting an index for fixing the examinee's gaze, and an observation optical system using infrared light and provide various complicated electronic components related thereto, and thus there is a problem that only an expert of the apparatus may take a high-quality fundus image.

### DETAILED DESCRIPTION OF THE INVENTION

### Technical Objectives

The present invention is directed to a fundus imaging device having an automatic focusing function by which even a user, who is not a skilled expert, may easily photograph a fundus image, and interpret and analyze the photographed image to predict a degree of risk.

### Technical Solution to the Problem

According to an embodiment, a fundus imaging device having an automatic focusing function includes: a body including a housing; and an imaging member disposed in the housing, wherein the imaging member includes: a first body having a first optical path therein; a second body having a second optical path to be inserted into a rear side of the first optical path, wherein the second body is movable forward or rearward along the first optical path; a first lens module positioned in front of the first optical path exposed to outside of the housing and guiding an examinee's eye; a second lens module positioned at a rear side of the second optical path; a light source positioned in the first optical path or the second optical path and emitting a light toward a direction of the examinee's eye; a pupil imaging unit provided in the first optical path and configured to photograph a pupil image of a light reflected from the examinee's eye; a fundus imaging unit provided at a rear side of the second lens module and configured to photograph a fundus image of the light reflected from the examinee's eye; a focusing unit configured to move the second body so that a distance between the first lens module and the second lens module becomes closer or farther away; and a controller configured to control the focusing unit to adjust a focus of the pupil image received from the pupil imaging unit and control the fundus imaging unit to photograph the fundus image when the pupil image is in focus.

In some embodiments, the light source may be provided in the first optical path or the second optical path and includes a first light source emitting an infrared light and a second light source emitting a white light, in the pupil imaging unit photographing the pupil image, the controller may turn on the first light source to control the infrared light to be directed in the direction of the examinee's eye, and in the fundus imaging unit photographing the fundus image, the controller may turn on the second light source to control the white light to be directed in the direction of the examinee's eye.

In some embodiments, the focusing unit may include: a drive motor fixed to the first body; a rotation shaft mounted on the drive motor and extending parallel to the second body; and a connector including one side connected to the second body and another side screw-coupled to the rotation shaft, and when the drive motor rotates the rotation shaft forward or reversely under control of the controller, the connector moves the second body to be closer to or farther away from the first lens module.

In some embodiments, the fundus imaging device having an automatic focusing function may further include: a support plate positioned outside the first body and the second body; a first support coupled to a front side of the support plate and including one side through which the first body passes and another side on which the drive motor is mounted; and a movement guide member coupled to a rear side of the support plate and including a second support through which a rear side of the second body passes, wherein when the connector connected to a front side of the second body moves the second body, the rear side of the second body slides in a state of being supported by the second support.

In some embodiments, the first optical path may include a (1-1)-st path, a (1-2)-nd path, and a (1-3)-rd path which are sequentially positioned, and the first lens module may be positioned in the (1-1)-st path, the pupil imaging unit is positioned in the (1-2)-nd path, and the second optical path is inserted in the (1-3)-rd path.

In some embodiments, the controller may be configured to control comparing and analyzing the fundus image received from the fundus imaging unit and pre-stored retinal-related disease data to interpret a level of risk, and outputting the interpreted result to the outside.

In some embodiments, the body may further include a base positioned below the housing, a 3-axis motor may be disposed between the base and the housing and configured to move the housing in directions of an X-axis, a Y-axis or a Z-axis, and the controller may control the 3-axis motor to move the housing so that the pupil image of the examinee received from the pupil imaging unit is located at a pre-interpreted fixation point.

In some embodiments, the fundus imaging device having an automatic focusing function may further include: a connection body unitarily formed to extend at one side of the first optical path, wherein a connection path communicating with the first optical path is formed in the connection body, a third lens module is provided in the connection path, and the pupil imaging unit is mounted on an outside of the third lens module positioned in a direction opposite to the first optical path.

In some embodiments, the fundus imaging device having an automatic focusing function may further include an input unit for transmitting an input signal to the controller, wherein when the controller receives a first input signal from the input unit, the controller may perform a first focusing mode to control the 3-axis motor, and when the controller receives a second input signal from the input unit, the controller may sequentially perform a second focusing mode to control the focusing unit and an output mode to output the fundus image received from the fundus imaging unit to the outside.

In some embodiments, the fundus imaging device having an automatic focusing function may further include a communication unit for transmitting data received from the controller to the outside through wireless communication.

### Effects of the Invention

The present invention has an effect that a pupil image photographed by a pupil imaging unit may be automatically positioned at a predetermined fixation point by controlling a 3-axis motor.

The present invention further has an effect that a pupil image may be automatically focused by controlling a focusing unit, and thus a fundus imaging unit may capture an optimal fundus image.

Furthermore, since a second body may safely move forward or rearward by using a movement guide member, the movement guide member may allow the focusing unit to guide the second body to move forward or rearward stably in the process of focusing the pupil image.

In addition, since a non-professional may easily take a fundus image and accurately interpret a retinal-related disease using the photographed fundus image, there is an effect of early treatment of a retinal-related disease.

### BRIEF DESCRIPTION OF DRAWINGS

FIG. 1 is a view schematically illustrating an external appearance of a fundus imaging device having an automatic focusing function according to an embodiment of the present invention.

FIG. 2 is a view illustrating an imaging member of a fundus imaging device having an automatic focusing function according to an embodiment of the present invention.

FIG. 3 is a vertical cross-sectional view illustrating an imaging member of a fundus imaging device having an automatic focusing function according to an embodiment of the present invention.

FIG. 4 is a horizontal cross-sectional view illustrating an imaging member of a fundus imaging device having an automatic focusing function according to an embodiment of the present invention.

FIG. 5 is a diagram illustrating a control means of a fundus imaging device having an automatic focusing function according to an embodiment of the present invention.

FIG. 6 is a diagram schematically illustrating a state in which a pupil image is positioned at a fixation point of a fundus imaging device having an automatic focusing function according to an embodiment of the present invention.

FIG. 7 is a view illustrating a propagation path of light emitted from a light source of a fundus imaging device having an automatic focusing function according to an embodiment of the present invention.

<Reference numeral>

| | |
|---|---|
| 100: body | 110: base |
| 112: seat portion | 120: housing |
| 130: 3-axis motor | 140: output unit |
| 200: imaging member | 210: first body |
| 212: first optical path | 212a: (1-1)-st path |
| 212b: (1-2)-nd path | 212c: (1-3)-rd path |
| 215: connection body | 216: connection path |
| 220: second body | 222: second optical path |
| 222a: (2-1)-st path | 222b: (2-2)-nd path |
| 230: first lens module | 232: second lens module |
| 232a: correction lens | 232b: image pickup lens |
| 234: third lens module | 236: beam splitter |
| 236a: first beam splitter | 236b: second beam splitter |
| 240: light source | 242: first light source |
| 244: second light source | 250: pupil imaging unit |
| 252: fundus imaging unit | 260: focusing unit |
| 262: drive motor | 264: rotation shaft |
| 266: connector | 270: movement guide member |
| 272: support plate | 274: first support |
| 276: second support | 300: control means |
| 301: input unit | 302: storage unit |
| 303: controller | 304: communication unit |
| 305: first light source driver | 306: second light source driver |
| 307: first motor driver | 308: second motor driver |

### MODES FOR CARRYING OUT THE INVENTION

Hereinafter, with reference to the accompanying drawings, a fundus imaging device having an automatic focusing function according to an embodiment of the present invention will be described in more detail.

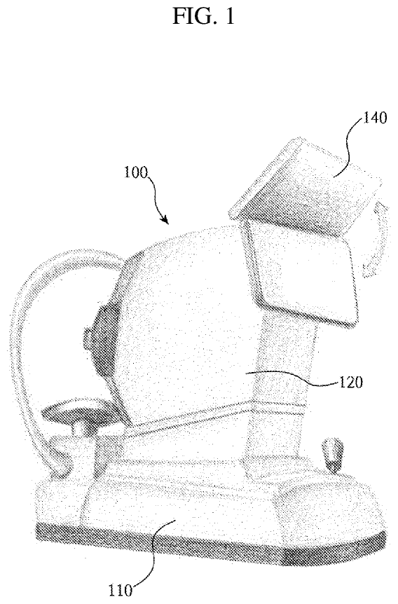

FIG. 1 is a view schematically illustrating an external appearance of a fundus imaging device having an automatic focusing function according to an embodiment of the present invention.

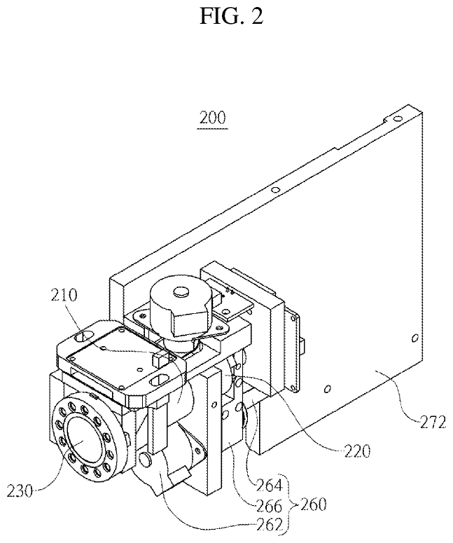

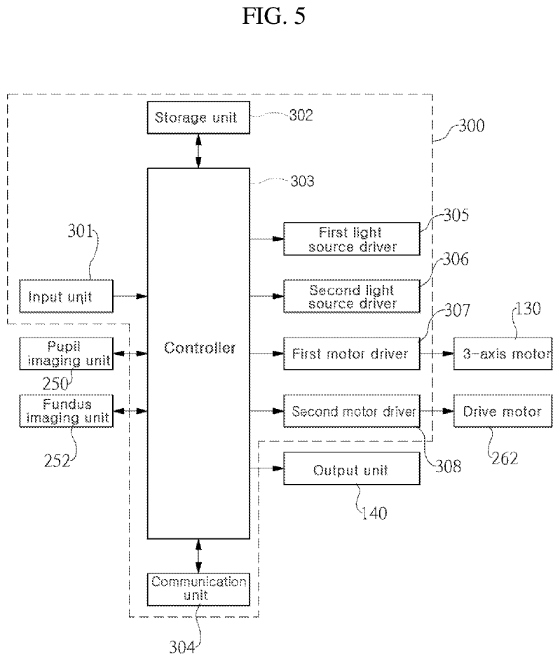

Referring to FIG. 1, a fundus imaging device (e.g., a fundus image photography apparatus) having an automatic focusing function according to an embodiment of the present invention includes a body (e.g., main body) 100, an imaging member 200 (see FIG. 2) and a control means 300 (see FIG. 5). The body 100 forms an appearance (e.g., an external shape) and includes a base 110 to be seated on the ground, a desk, and the like; a housing 120 positioned on the base 110 and defining a space so that various parts may be disposed therein; a 3-axis motor 130 (see FIG. 5) positioned between the base 110 and the housing 120; and an output unit 140 positioned on the housing 120. A seat portion 112 is provided in front of (e.g., at the front of) the base 110 so that a chin of an examinee is seated.

The 3-axis motor 130 may move the housing 120 in three axis directions, e.g., an X-axis, a Y-axis, and a Z-axis, in a state of being supported by the base 110. In such an embodiment, the Z-axis refers to a front-rear direction moving away from or closer to the seat portion 112 with respect to the seat portion 112, the X-axis refers to a vertical direction of the front-rear direction, that is, a left-right direction, and the Y-axis refers to an up-down direction. Hereinafter, unless otherwise specified, the term “front” or “frontward” or “front end” may refer to a direction closer to the seat portion 112, and the term “rear” or “rearward” or “rear end” may refer to a direction further away from the seat portion 112.

The output unit 140 may include a conventional touch panel and the like capable of outputting various images to the outside and may be rotatably coupled to a rear side of the housing 120. The output unit 140 may display an image of a current progress and an examiner interface UI screen for performing a function. In addition, the output unit 140 is designed to be rotatable upward so that the examiner may easily operate it.

The imaging member 200 and the control means 300 are positioned in the housing 120 and will be described below.

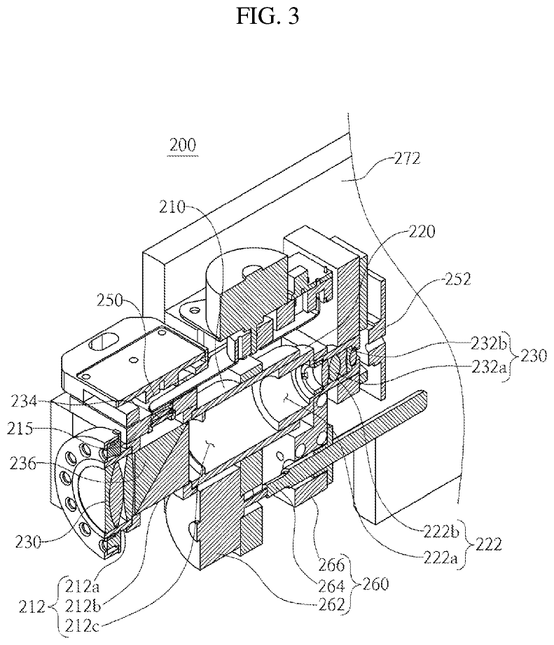

FIG. 2 is a view illustrating an imaging member of a fundus imaging device having an automatic focusing function according to an embodiment of the present invention, and FIG. 3 is a vertical cross-sectional view illustrating an imaging member of a fundus imaging device having an automatic focusing function according to an embodiment of the present invention.

Referring to FIGS. 2 and 3, the imaging member 200 is provided in the housing 120 to photograph an eye of an examinee, and includes a first body 210, a first lens module 230, a beam splitter 236, a connection body 215, a pupil imaging unit 250, a second body 220, a second lens module 232, a fundus imaging unit 252, a light source 240, a focusing unit (e.g., focus adjustor) 260, and a movement guide member 270.

The first body 210 is formed, for example, in a cylindrical shape, and a first optical path 212 is provided therein to define an elongated empty space so that light may pass therethrough. In the first optical path 212, a (1-1)-st path 212a, a (1-2)-nd path 212b, and a (1-3)-rd path 212c are sequentially positioned from a front side to a rear side along a longitudinal direction thereof. The front side of the (1-1)-st path 212a is exposed to the outside through the front side of the housing 120, and the examinee's eye is positioned in front of the (1-1)-st path 212a exposed to the outside. The first lens module 230 is positioned in the (1-1)-st path 212a, and the beam splitter 236 is positioned in the (1-2)-nd path 212b.

The first lens module 230 includes a plurality of objective lenses, and the objective lenses have, for example, a conventional configuration for extending a light path, adjusting a size of light, or adjusting a focus. The first lens module 230 is disposed in the (1-1)-st path 212a exposed to the outside of the housing 120, and the examinee's eye may be guided to the first lens module 230.

The beam splitter 236 transmits some light, while reflecting some other light, and includes a first beam splitter 236a and a second beam splitter 236b positioned in the second path in an up-down direction.

The connection body 215 is unitarily formed extending upwardly of an upper side of the first body 210, in detail, an upper side of the (1-2)-nd path 212b. A connection path 216 communicating with the (1-2)-nd path 212b is formed in the connection body 215, and the third lens module 234 is positioned in the connection path 216. The third lens module 234 includes a plurality of objective lenses, and adjusts size, focus, and the like of a pupil image formed on the pupil imaging unit 250 to be described below.

The pupil imaging unit 250 is mounted on the connection body 215, and specifically, is mounted on an outside of the third lens module 234 located in a direction opposite to the (1-2)-nd path 212b. The pupil imaging unit 250 is a camera for photographing a pupil image of the examinee, and may include, for example, a charge coupled device (CCD) module or a complementary metal oxide semiconductor (CMOS) module capable of sensitively accepting a near-infrared light in a range from about 740 to 780 nm. The pupil image photographed by the pupil imaging unit 250 may be transmitted to the controller 303 to be described below, and the controller 303 may output the pupil image photographed by the pupil imaging unit 250 to the output unit 140 so that a position of the examinee's pupil may be tracked and observed.

The second body 220 is formed in a long tubular shape to be coupled to a rear side of the first body 210, and a second optical path 222 is defined therein to be inserted into the (1-3)-rd path 212c. The second body 220 may be movable forward or rearward along the (1-3)-rd path 212c. The second optical path 222 includes a (2-1)-st path 222a inserted into the (1-3)-rd path 212c and a (2-2)-nd path 222b unitarily extending at a rear side of the (2-1)-st path 222a. The (2-2)-nd path 222b is formed to have a smaller diameter than the (2-1)-st path 222a, and a second lens module 232 is provided therein.

The second lens module 232 includes a plurality of correction lenses 232a for transmitting an image as an image of a desired condition; and an image pickup lens 232b positioned at a rear side of the correction lens 232a.

The fundus imaging unit 252 photographs an image formed on the image pickup lens 232b and is provided at a rear side of the second lens module 232 to photograph an image of light reflected from the examinee's eye. The fundus imaging unit 252 may include a charge coupled apparatus (CCD) module or a complementary metal oxide semiconductor (CMOS) module capable of sensitively receiving a white light in a range of about 450 to 780 nm.

The light source 240 which is positioned in the first optical path 212 or the second optical path 222, preferably in the (2-1)-st path 222a, may emit light in a direction of the examinee's eye. The light source 240 includes a first light source 242 and a second light source 244. The first light source 242 is an illumination used when observing and tracking an eyeball and emits a near-infrared light in a range of about 740 to 780 nm in the direction of the examinee's eye. When a near-infrared light in a range of 740 to 780 nm is directed to the examinee's eye, the pupil of the examinee is not dilated, such that the pupil of the examinee may be easily observed. The second light source 244 is an illumination used when photographing a fundus image and emits a white light in a range of about 450 to 780 nm in the direction of the examinee's eye. That is, a white light is instantaneously emitted such that a visible light is incident on the fundus through a dilated pupil, and a fundus image is acquired from light reflected from the fundus. The pupil image taken by using the first light source 242 is not image data suitable as a basis for fundus examination, but it is an image for automatically adjusting the focus of the pupil. The fundus image taken by using the second light source 244 is image data for fundus examination, and various fundus examinations are performed using it.

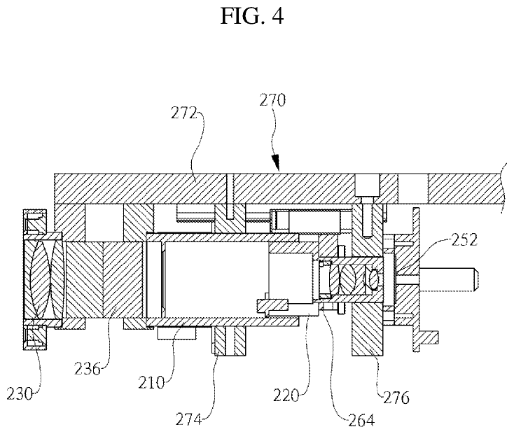

The focusing unit 260 and the movement guide member 270 will be described with reference to FIG. 4.

FIG. 4 is a horizontal cross-sectional view illustrating an imaging member of a fundus imaging device having an automatic focusing function according to an embodiment of the present invention.

Referring to FIGS. 3 and 4, the focusing unit 260 moves the second body 220 so that a distance between the first lens module 230 and the second lens module 232 becomes closer to or farther away from each other so as to obtain more accurate and clear pupil and fundus images, and includes a drive motor 262 fixed to the first body 210; a rotation shaft 264 mounted on the drive motor 262 and extending parallel to the second body 220; and a connector 266 having one side connected to the second body 220 and another side screw-coupled to the rotation shaft 264. In an embodiment, a separate control terminal (not illustrated) may be provided to drive the drive motor 262. The rotation shaft 264 is formed to be elongated in a longitudinal direction and is configured to be rotated by rotation of the drive motor 262. The connector 266 is configured to allow the rotation shaft 264 to pass therethrough and is screw-coupled to the rotation shaft 264, thereby converting a rotational motion of the rotation shaft 264 into a linear motion. Accordingly, when the drive motor 262 rotates the rotation shaft 264 forward or reversely under control of the controller 303 to be described below, the connector 266 moves the second body 220 to be closer to or farther away from the first lens module 230 so that the pupil image photographed by the pupil imaging unit 250 is focused.

The movement guide member 270 guides movement of the second body 220 and includes a support plate 272 positioned outside the first body 210 and the second body 220; a first support 274 coupled to a front side of the support plate 272 and including one side through which the first body 210 passes and another side on which the drive motor 262 is mounted; and a second support 276 coupled to a rear side of the support plate 272 and through which a rear side of the second body 220 passes.

When the connector 266 connected to the front side of the second body 220 moves the second body 220, the rear side of the second body 220 slides forward or rearward in a state of being supported by the second support 276. As such, the present invention has the effect of allowing safe movement of the second body 220 forward or rearward by using the movement guide member 270.

FIG. 5 is a diagram illustrating a control means of a fundus imaging device having an automatic focusing function according to an embodiment of the present invention.

Referring to FIG. 5, the controller 300 includes an input unit 301, a storage unit 302, a controller 303, a communication unit 304, a first light source driver 305, a second light source driver 306, a first motor driver 307 and a second motor driver 308.

The input unit 301 is mounted on the body 100, receives various input signals from the outside, and transmits them to the controller 303. The input unit 301 may be separately formed in the body 100 or may be formed in the output unit 140 in the form of a touch pad.

The storage unit 302 stores the fundus image photographed by the fundus imaging unit 252 as an image file. The communication unit 304 transmits various data, such as an image file received from the controller 303, to an external terminal through wireless communication. The wireless communication may include, for example, common short-distance wireless communication such as Bluetooth, NFC, WiFi, or the like, or common long-distance wireless communication such as 3G, 4G, and 5G.

The first light source driver 305 drives the first light source 242 under control of the controller 303, and the second light source driver 306 drives the second light source 244 under control of the controller 303. The first motor driver 307 drives the 3-axis motor 130 under control of the controller 303, and the second motor driver 308 drives the drive motor 262 under control of the controller 303.

When the controller 303 receives a first input signal from the input unit 301, the controller 303 performs a first focusing mode to control the 3-axis motor 130, and when the controller 303 receives a second input signal from the input unit 301, the controller 303 sequentially performs a second focusing mode to control the focusing unit 260, an output mode to output the fundus image received from the fundus imaging unit 252 to the outside, and an interpretation mode to analyze the fundus image to interpret a level of risk. Hereinafter, the first focusing mode, the second focusing mode, the output mode, and the interpretation mode will be described.

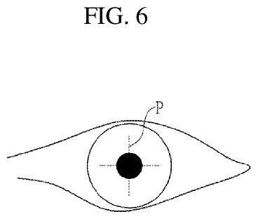

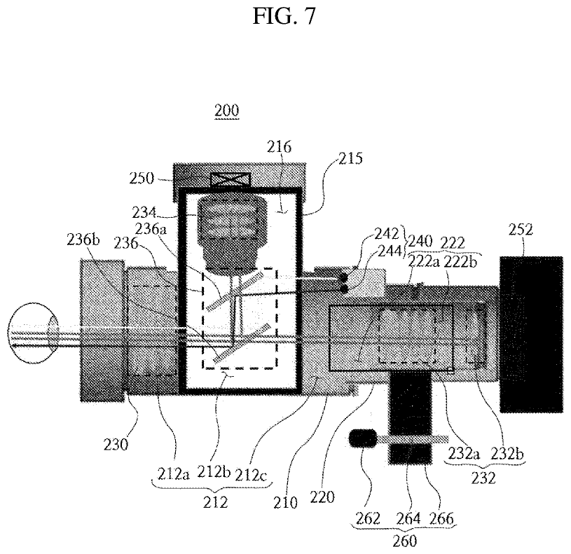

FIG. 6 is a diagram schematically illustrating a state in which a pupil image is positioned at a fixation point of a fundus imaging device having an automatic focusing function according to an embodiment of the present invention, and FIG. 7 is a view illustrating a propagation path of light emitted from a light source of a fundus imaging device having an automatic focusing function according to an embodiment of the present invention.

Referring to FIGS. 1 to 7, the controller 303 sequentially performs the first focusing mode, the second focusing mode, the output mode, and the interpretation mode.

First, the first focusing mode is for adjusting the focus of the pupil, and in the first focusing mode, an examinee's chin is firstly seated on the seat portion 112, and then an examinee's eye is positioned at the first lens module 230. In such a state, the controller 303 receives the first input signal from the input unit 301. Then, the controller 303 drives the first light source driver 305 so that the first light source 242 emits light. Then, the first light source 242 is turned on to emit a near-infrared light from the first light source 242, and the near-infrared light passes through the first beam splitter 236a and then is guided in a direction toward the first lens module 230 from the second beam splitter 236b. The near-infrared light reflected from the examinee's eye located at the first lens module 230 is directed toward the second beam splitter 236b through the first lens module 230, and in such a case, some of the near-infrared light is reflected in a direction toward the first beam splitter 236a from the second beam splitter 236b and then is directed toward the pupil imaging unit 250 via the third lens module 234, and some of the near-infrared light passes through the second beam splitter 236b and is directed toward the fundus imaging unit 252 via the second lens module 232. The pupil imaging unit 250 photographs the pupil of the eye directed to the third lens module 234 and transmits the photographed pupil image to the controller 303. Then, the controller 303 controls the first motor driver 307, thereby controlling the 3-axis motor 130 to move the housing 120 in 3-axis directions, for example, an X-axis, a Y-axis, and a Z-axis. Then, the pupil image of the examinee received from the pupil imaging unit 250 in FIG. 6 is controlled to be positioned at a predetermined fixation point P, for example, a reference position.

The fixation point P may be configured in a conventional “+” shape. In such a case, since the pupil image and the predetermined fixation point P are imaged together on the external output unit 140, the examiner may check whether the pupil image is properly aligned at the fixation point P. As such, the present invention has the effect of automatically positioning the pupil image photographed by the pupil imaging unit 250 at the predetermined fixation point P by controlling the 3-axis motor 130. In an embodiment, if the pupil image is not properly aligned with the fixation point P despite the 3-axis motor 130 moving the housing 120, the examiner may manually control the 3-axis motor 130 by using the input unit 301 such that the pupil image of the examinee is aligned at the predetermined fixation point P.

Next, the second focusing mode is for controlling the focus of the pupil image of the examinee received from the pupil imaging unit 250 to be accurately focused, and in the second focusing mode, the controller 303 checks whether the pupil image of the examinee received from the pupil imaging unit 250 is in focus. Then, the controller 303 controls the second motor driver 308, thereby controlling the drive motor 262 of the focusing unit 260 to move the second body 220 in the front-rear direction to focus the pupil image (e.g., to be in focus). That is, when the pupil image of the examinee received from the pupil imaging unit 250 is controlled to be in focus, the fundus imaging unit 252 to be described below may take a more accurate, clear, and optimal fundus image, so it is controlled such that the pupil image is accurately focused in advance before the fundus imaging unit 252 photographs the fundus image. As such, in the present invention, the focusing unit 260 is controlled so that the pupil image is automatically focused, and thus the fundus imaging unit 252 may photograph the optimal (e.g., best) fundus image.

Next, in the output mode, in a state in which the pupil image is firstly controlled to be focused, the controller 303 receives the second input signal from the input unit 301. Then, the controller 303 controls the first light source driver 305 and the second light source driver 306 so that the first light source 242 is turned off and the second light source 244 is turned on for a short time. Then, the second light source 244 sequentially passes through the first beam splitter 236a and the second beam splitter 236b and then is directed to the examinee's pupil through the first lens module 230. Accordingly, the examinee's pupil is instantly dilated (e.g., widened), and at this time, a fundus image, that is, a retinal image is obtained from light reflected back to the fundus imaging unit 252.

Then, in the interpretation mode, firstly, the controller 303 stores the received retina image in the storage unit 302. Then, the storage unit 302 compares and analyzes the retinal image and retina-related disease data pre-stored in the storage unit 302 to interpret a level of risk, and outputs the interpreted result to the outside. In such a case, the controller 303 may continuously develop the interpretation performance by repeatedly performing machine learning using a deep learning-based artificial intelligence technique.

As such, in the present invention, even a user who is not a skilled expert may easily take a fundus image and accurately interpret (e.g., read) retinal-related diseases using the photographed fundus images, thereby achieving the effect of early treatment of retinal-related diseases.

Furthermore, since the image taken by the pupil imaging unit 250 is used in the process of focusing, and the image photographed by the fundus imaging unit 252 is used in photographing the retina image, the apparatus may be set so that the pupil imaging unit 250 is optimized to focusing and the fundus imaging unit 252 is optimized to photographing a retina image. Accordingly, the apparatus may be set such that functions of the pupil imaging unit 250 and the fundus imaging unit 252 may be best achieved according to each purpose.

Although the present invention has been described in detail in the above embodiments, the present invention is not limited thereto, and it is apparent to those skilled in the art that various changes and modifications may be made within the scope of the technical spirit of the present invention. If such variations and modifications fall within the scope of the appended claims, such technical idea should also be regarded as belonging to the present invention.

What is claimed is:

1. A fundus imaging device having an automatic focusing function, comprising:
a body including a housing; and
an imaging member disposed in the housing,
wherein the imaging member comprises:
a first body having a first optical path therein;
a second body having a second optical path to be inserted into a rear side of the first optical path, wherein the second body is movable forward or rearward along the first optical path;
a first lens module positioned in front of the first optical path exposed to outside of the housing and guiding an examinee's eye;
a second lens module positioned at a rear side of the second optical path;
a light source positioned in the first optical path or the second optical path and emitting a light toward a direction of the examinee's eye;
a pupil imaging unit provided in the first optical path and configured to photograph a pupil image of a light reflected from the examinee's eye;
a fundus imaging unit provided at a rear side of the second lens module and configured to photograph a fundus image of the light reflected from the examinee's eye;
a focusing unit configured to move the second body so that a distance between the first lens module and the second lens module becomes closer or farther away; and
a controller configured to control the focusing unit to adjust a focus of the pupil image received from the pupil imaging unit and control the fundus imaging unit to photograph the fundus image when the pupil image is in focus.

2. The fundus imaging device having an automatic focusing function of claim 1,
wherein the light source is provided in the first optical path or the second optical path and comprises a first light source emitting an infrared light and a second light source emitting a white light,
in the pupil imaging unit photographing the pupil image, the controller turns on the first light source to control the infrared light to be directed in the direction of the examinee's eye, and
in the fundus imaging unit photographing the fundus image, the controller turns on the second light source to control the white light to be directed in the direction of the examinee's eye.

3. The fundus imaging device having an automatic focusing function of claim 1,
wherein the focusing unit comprises:
a drive motor fixed to the first body;
a rotation shaft mounted on the drive motor and extending parallel to the second body; and
a connector comprising one side connected to the second body and another side screw-coupled to the rotation shaft, and
when the drive motor rotates the rotation shaft forward or reversely under control of the controller, the connector moves the second body to be closer to or farther away from the first lens module.

4. The fundus imaging device having an automatic focusing function of claim 3, further comprising:
a support plate positioned outside the first body and the second body;
a first support coupled to a front side of the support plate and comprising one side through which the first body passes and another side on which the drive motor is mounted; and
a movement guide member coupled to a rear side of the support plate and comprising a second support through which a rear side of the second body passes,
wherein when the connector connected to a front side of the second body moves the second body, the rear side of the second body slides in a state of being supported by the second support.

5. The fundus imaging device having an automatic focusing function of claim 1,
wherein the first optical path comprises a (1-1)-st path, a (1-2)-nd path, and a (1-3)-rd path which are sequentially positioned, and
the first lens module is positioned in the (1-1)-st path, the pupil imaging unit is positioned in the (1-2)-nd path, and the second optical path is inserted in the (1-3)-rd path.

6. The fundus imaging device having an automatic focusing function of claim 1,
wherein the controller is configured to control comparing and analyzing the fundus image received from the fundus imaging unit and pre-stored retinal-related disease data to interpret a level of risk, and outputting the interpreted result to the outside.

7. The fundus imaging device having an automatic focusing function of claim 1,
wherein the body further comprises a base positioned below the housing,
a 3-axis motor is disposed between the base and the housing and configured to move the housing in directions of an X-axis, a Y-axis or a Z-axis, and
the controller controls the 3-axis motor to move the housing so that the pupil image of the examinee received from the pupil imaging unit is located at a pre-interpreted fixation point.

8. The fundus imaging device having an automatic focusing function of claim 7,
further comprising a connection body unitarily formed to extend at one side of the first optical path,
wherein a connection path communicating with the first optical path is formed in the connection body,
a third lens module is provided in the connection path, and
the pupil imaging unit is mounted on an outside of the third lens module positioned in a direction opposite to the first optical path.

9. The fundus imaging device having an automatic focusing function of claim 7,
further comprising an input unit for transmitting an input signal to the controller,
wherein when the controller receives a first input signal from the input unit, the controller performs a first focusing mode to control the 3-axis motor, and when the controller receives a second input signal from the input unit, the controller sequentially performs a second focusing mode to control the focusing unit and an output mode to output the fundus image received from the fundus imaging unit to the outside.

10. The fundus imaging device having an automatic focusing function of claim 1,
further comprising a communication unit for transmitting data received from the controller to the outside through wireless communication.
